(19) United States
(12) Patent Application Publication
(10) Pub. No.: US 2007/0055319 A1
(43) Pub. Date: Mar. 8, 2007

(54) Connector module replacement for implantable medical stimulators

(75) Inventor: Scott A. Spadgenske, Fridley, MN (US)

Correspondence Address: NIKOLAI & MERSEREAU, P.A., 900 SECOND AVENUE SOUTH, SUITE 820, MINNEAPOLIS, MN, 55402, US

(73) Assignee: Cardiac Pacemakers, Inc., St. Paul, MN (US)

(21) Appl. No.: 11/580,753

(22) Filed: Oct. 13, 2006

Related U.S. Application Data
(62) Division of application No. 10/222,145, filed on Aug. 16, 2002, now abandoned.

(51) Int. Cl. A61N 1/375

(52) U.S. Cl. 607/37; 607/9

(57) ABSTRACT

An adapter module for use with a headerless pulse generator of implantable tissue stimulator includes a first connector member at one end for mating with contact pins of a feed-through assembly of the pulse generator and medical lead receiving contacts at a second end thereof for mating with proximal terminal pins of one or more medical leads used to apply stimulating pulses to target tissue. Use of the adapter obviates the need to have a particular pulse generator model for every type of lead that may be encountered at the time of implant.

### CROSS REFERENCE TO RELATED APPLICATION

This application is a divisional of application Ser. No. 10/222,145, filed Aug. 16, 2002, and entitled “Connector Module Replacement for Implantable Medical Stimulators”.

### BACKGROUND OF THE INVENTION

I. Field of the Invention

This invention relates generally to implantable medical stimulating apparatus, and more particularly to an adapter for interfacing a product line of different implantable pulse generators to a variety of medical leads such that the need for a hospital or clinic to inventory many different pulse generator models in stock for each type of lead connector that might be encountered, say, in a pulse generator replacement procedure, is eliminated.

II. Discussion of the Prior Art

The major suppliers of implantable medical devices, such as automatic implantable cardiac defibrillators (AICDs) and pacemakers for treating bradycardia, tachycardia and congestive heart failure, offer a number of different models, each with its own style of header having lead terminal bores with electrical contacts for mating with a variety of lead types. At a time that it becomes necessary to replace a stimulation pulse generator due to battery depletion or other reasons, a hospital or clinic must have, on hand, an appropriate model that can be used with the existing lead configuration extant in the patient. When it is considered that there are at least five different proximal lead connectors to be accommodated and given the significant number of different pulse generator models having headers that must mate with one or more of the possible proximal lead connector configurations, a significant inventory of relatively expensive pulse generators must be maintained due to connector differences.

There is a need, then, for an adapter having a first connector capable of mating with a plurality of different pulse generators and a second connector capable of mating with the proximal terminal pin on one or more medical leads such that it is only necessary to maintain a small stock of relatively inexpensive adapters that allow the physician to operatively mate a pulse generator to leads having a given proximal lead connector type. With such adapters available, the number of expensive pulse generator models that must be maintained in an inventory can be significantly reduced. Rather than having one model number for each type of lead connector that may be encountered during a device upgrade or exchange, a single pulse generator device can be stocked with a family of adapters. The adapters all have one common connector that mates up with the feed-through pins of the pulse generator. This provides commonality across all products, leading to reduced costs. Further, when a new generation of pulse generators is being designed, there would be no need to redesign all of the headers of these pulse generators to fit a new device shape. It would only be necessary for the clinic to stock a family of low cost adapters rather than an entire family of pulse generators, some of which may never be used or whose shelf life can expire before put in use.

### SUMMARY OF THE INVENTION

In accordance with the present invention there is provided an adapter for permitting the coupling of an implantable cardiac rhythm management device to a variety of stimulating lead connector types, the cardiac rhythm management device comprising a housing containing an electronic circuit and a feed-through assembly hermetically sealing the housing and having a plurality of contact pins connected to the electronics circuit. The adapter comprises a first connector having contacts that are adapted to mate directly with the plurality of contact pins of the feed-through assembly and a second connector having contacts adapted to mate with at least one of a plurality of proximal connector pin types employed on cardiac stimulating leads, the contacts of the first and second connectors are joined in a predetermined manner by conductors internal to the adapter. In particular, the second connector has contacts that are adapted to mate with at least one type of cardiac stimulating lead whose proximal terminal pin meet International Standards. Persons skilled in the art relating to leads for implantable cardiac rhythm management devices are familiar with the configuration and dimensions of the proximal connectors meeting existing and currently proposed International Standards, e.g., IS-1, IS-4, DF-1 and possibly other yet not-standard connectors, e.g. IS-4 (PC-73).

### DESCRIPTION OF THE DRAWINGS

The foregoing features, objects and advantages of the invention will become apparent to those skilled in the art from the following detailed description of a preferred embodiment, especially when considered in conjunction with the accompanying drawings in which like numerals in the several views refer to corresponding parts.

FIG. 1 is a perspective view of an adapter module for interfacing a plurality of medical leads having differing proximal connector types with a pulse generator module;

FIG. 2 is a bottom plan view of the adapter of FIG. 1; and

FIG. 3 is a perspective view of an alternative embodiment.

### DESCRIPTION OF THE PREFERRED EMBODIMENT

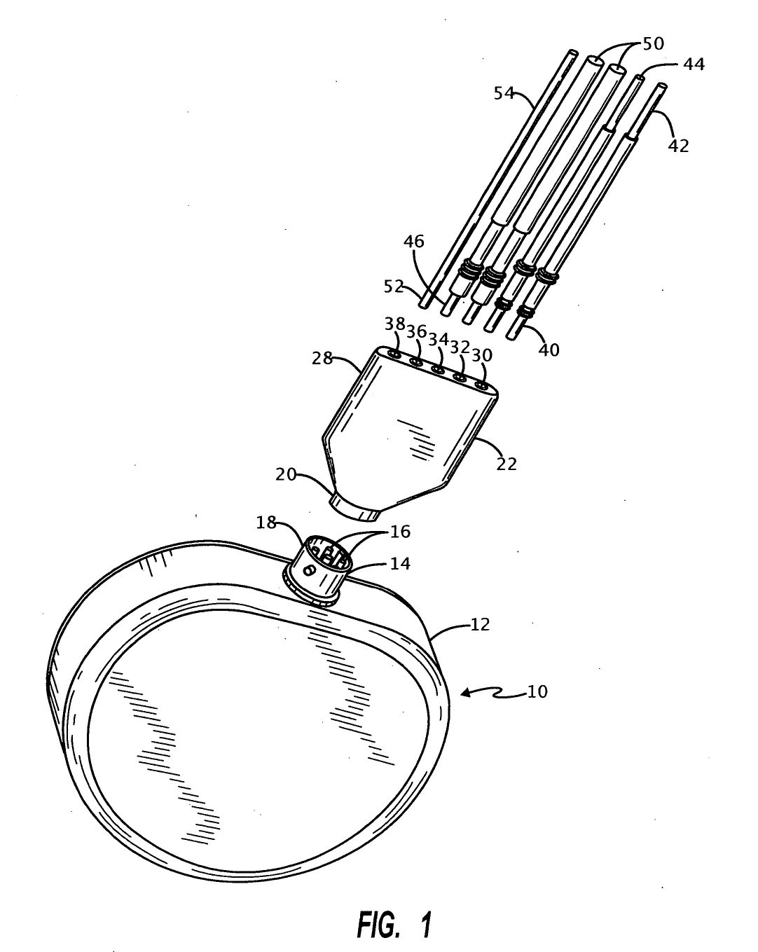

Referring first to FIG. 1, there is shown an implantable medical device, such as a cardiac rhythm management device or a neural stimulator and it is indicated generally by numeral 10. The implantable device comprises a hermetically sealed housing 12, preferably fabricated from titanium and which is commonly referred to as the “can”. As is known in the art, contained within the can is a battery power supply and a pulse generator that is controlled by a programmed microprocessor to deliver stimulating pulses at timed intervals determined by the program executed by the microprocessor. Input signals to the electronics module within the can 12 and output signals there from are provided by way of a feed-through assembly 14 which includes an insulating base (not shown) and a plurality of conductive contact pins as at 16 that pass through seals in the base member. The pins 16 connect to predetermined circuit nodes of the pulse generator electronics. An annular collar 18 surrounds the feed-through assembly 14 and is configured to mate with sockets of a female connector assembly 20 of an adapter module 22.

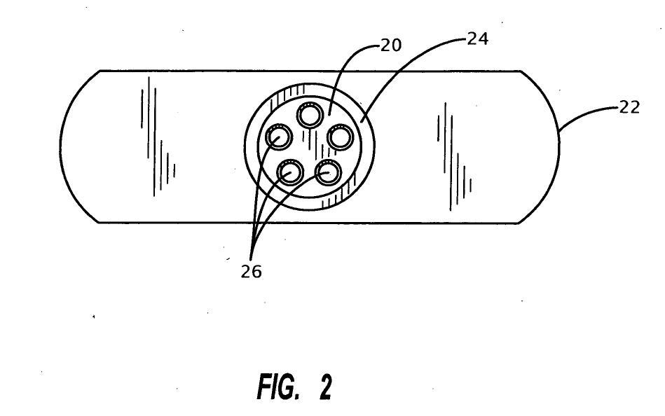

As seen in FIG. 2, the annular collar 28 surrounding the feed-through 14 on the pulse generator's can 12 is adapted to fit within the annular collar 24 of the female connector assembly 20 and the feed-through pins 16 plug into the sockets containing female contacts, as at 26 of the adapter 22. In the embodiment of FIG. 1, the adapter comprises a plurality of conductors (not shown) embedded in a plastic body 28 which extend individually from the female contacts 26 at a first end of the adapter to predetermined lead sockets 30, 32, 34, 36 and 38 formed longitudinally in the adapter body 28. The lead barrels 30-38 contain contacts that are adapted to mate with a proximal terminal pin and possibly a proximal terminal ring of a medical lead, depending upon the type of lead involved. For purposes of illustration only, the adapter 22 shown in FIG. 1 is illustrated as having five lead barrels 30, 32, 34, 36 and 38. Lead terminal barrels 30 and 32 may be designed to accommodate a medical lead having an IS-1 type proximal pin terminal 40 shown as being on the distal end portion of a medical leads 42 and 44. The lead barrel 34 can be designed to accommodate a proximal lead terminal pin 46 or a terminal pin 48 disposed on a DF-1 type defibrillating lead. Similarly, the lead barrels 34 and 36 are also configured to mate with a DF-1 lead 50. Finally, the lead barrel 38 of the adapter 22 is specifically designed to mate to with the proximal pin connector 52 on a small diameter, (LV-1 type) left ventricular lead (LV-1) 54, a proprietary design of Guidant Corporation, the assignee of the present invention.

To take full advantage of the flexibility afforded by the present invention, all of the medical devices of differing types offered by a particular supplier will have identical feed-through assemblies 14, even though one or more of the pins 16 may be a dummy, meaning that it is not internally connected to an active circuit node within the can 12. This uniformity in feed-through configuration, however, allows any one of several adapters 22 to mate with every pulse generator in the family. Such a family may include pulse generators designed to treat bradycardia, pulse generators to treat tachycardia, pulse generators to treat congestive heart failure and in each of such applications, there may be more than one type. For example, there are single chamber devices, dual chamber devices, rate adaptive devices, etc. By using a selected one of the adapters of the present invention, an implanting doctor can take note of the type of leads involved and then match that particular set of stimulating leads to the pulse generator by selecting an appropriate adapter.

In the event that a particular patient requires less than all of the leads that can be mated to a particular adapter and adapters with an equivalent number bores is not available, unused lead bores can be plugged so as to remain unused.

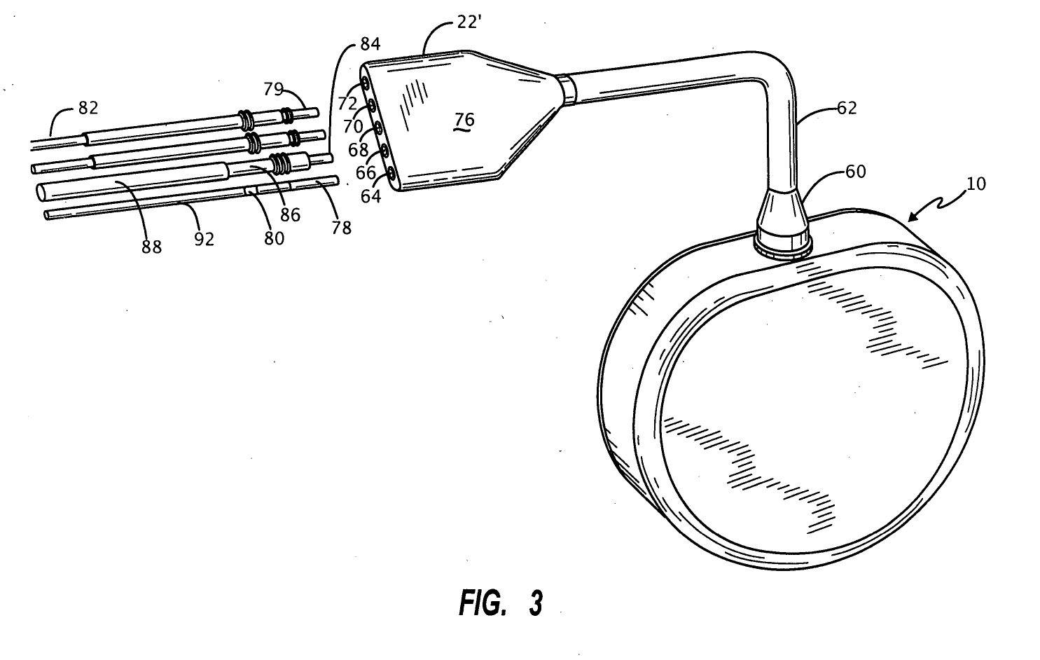

FIG. 3 shows an alternative embodiment of an adapter for use in mating medical leads having differing proximal connectors to any one of a number of implantable tissue stimulating devices. As in the embodiment of FIG. 1, the pulse generator 10 includes a feed-through assembly like that described in the Flynn et al. U.S. Pat. No. 5,906,634, the teachings of which are hereby incorporated by reference as if set forth in full herein. Beneath the elastomeric boot 60 is a connector, which may be of the type shown in FIG. 2 hereof or, alternatively, any one of the types set out in the Flynn et al. '634 patent for mating with the particular style of feed-through utilized on the pulse generator. The connector beneath the boot 60 is attached by insulated conductors within a short length of flexible cabling 62 to contacts within the bores 64-72 formed in a connector block 76 that is affixed to the other end of the cabling 62.

The adapter assembly 22′ has its lead barrel contacts arranged to mate with the proximal lead terminals of predetermined lead types. Thus, the contacts (not shown) contained within the lead barrel 64 may be positioned so as to engage the lead terminal pin 78 and lead ring contact 80 of a proprietary lead 92. Similarly, the bore or lead barrel 72 may have contacts positioned to mate with the lead terminal pin 79 and ring contact 86 of a lead 82 having a Type IS-1 terminal thereon. A further adapter bore, as at 68, may be designed to mate with the terminal 84 of a DF-1 defibrillator lead 88. By providing the cable 62, the adapter can be wrapped about the pulse generator can 10 and placed in the same surgically created pocket as the pulse generator itself.

With either the embodiments of FIGS. 1 and 3, the pulse generators 10 may be shipped from the manufacturer to a hospital or clinic with multiple versions of the adapter that have differing multi-lead branch ends to interface with any one of a variety of indwelling leads. The connector 20 on the adapter 22 (FIGS. 1 and 2) comprises a connector that would be common for all pulse generator models produced by the manufacturer and would be connected to the pulse generator at the time of implant and permanently fixed. The opposite, multi-lead end of the adapter allows lead terminals to be removably mated to the pulse generator, via the adapter module 22.

This invention has been described herein in considerable detail in order to comply with the patent statutes and to provide those skilled in the art with the information needed to apply the novel principles and to construct and use such specialized components as are required. However, it is to be understood that the invention can be carried out by specifically different equipment and devices, and that various modifications, both as to the equipment and operating procedures, can be accomplished without departing from the scope of the invention itself.

What is claimed is:

1-16. (canceled)

17. A method for mating any one of a plurality of implantable pulse generator models to medical leads present in a patient at the time of pulse generator replacement comprising the steps of:
(a) providing a headerless pulse generator having exposed feed-through contacts arranged in a predetermined pattern;
(b) providing an adapter comprising an insulating body having first and second ends, the first end having female sockets containing electrical contacts arranged in said predetermined pattern and the second end having a plurality of female sockets with contacts conforming to contacts present on proximal male terminal pins of each ISO standard medical lead for implantable medical devices;
(c) plugging the female sockets on the first end of the adapter onto the exposed feed-through contacts of a selected pulse generator model; and
(d) plugging the proximal male terminal pins of the medical leads present in the patient into the ones of the female sockets on the second end of the adapter appropriate to the ISO standard medical lead present in the patient.
